(19) United States
(12) Plant Patent Application Publication
(10) Pub. No.: US 2002/0120967 P1
(43) Pub. Date: Aug. 29, 2002

(54) Autumn Byrd raspberry variety

(76) Inventors: Elizabeth Keep, East Malling (GB); Victoria Hamilton Knight, West Malling (GB)

Correspondence Address: ROBERT B. HUGHES, HUGHES & SCHACHT, P.S., 2801 MERIDIAN STREET, SUITE 1, BELLINGHAM, WA, 98225-2412, US

(21) Appl. No.: 09/795,946

(22) Filed: Feb. 27, 2001

(51) Int. Cl. A01H 5/00

(52) U.S. Cl. PLT/204

(57) ABSTRACT

The present invention is a third backcross from Rubus spectabilis and has Rubus arcticus, Rubus odoratus, Rubus idaeus strigosus and Rubus crataegifolius in its background it is the first raspberry variety that has Rubus spectabilis in addition to the four aforementioned species in its background.

### FIELD OF THE INVENTION

The invention relates to raspberry plant varieties.

### SUMMARY OF THE INVENTION

To the present invention is a new a distinct variety of raspberry plant named “Autumn Byrd”. Autumn Byrd is a third backcross from Rubus spectabilis. It also has Rubus arcticus, Rubus odoratus, Rubus idaeus strigosus and Rubus crataegifolius in its background it is the first raspberry variety that has Rubus spectabilis in addition to the four aforementioned species in its background.

### BRIEF DESCRIPTION OF THE DRAWINGS

The following photographs illustrate the plant that is subject to the present invention where the colors being as true as can reasonably be done by conventional photographic procedures.

FIG. 1 is a view of the fruit of the plants of the present invention;

FIG. 2 is a view of the cane and leaves;

FIG. 3 is a close-up view of another item of the fruit;

FIG. 4 is a close-up view of the stock further showing the leaves and foliage of the present invention;

FIG. 5 is a second photographic view of the central portion of the stock further showing the leaves and foliage;

FIG. 6 is a top view of the leaves and foliage of the plants that is subject to the present invention;

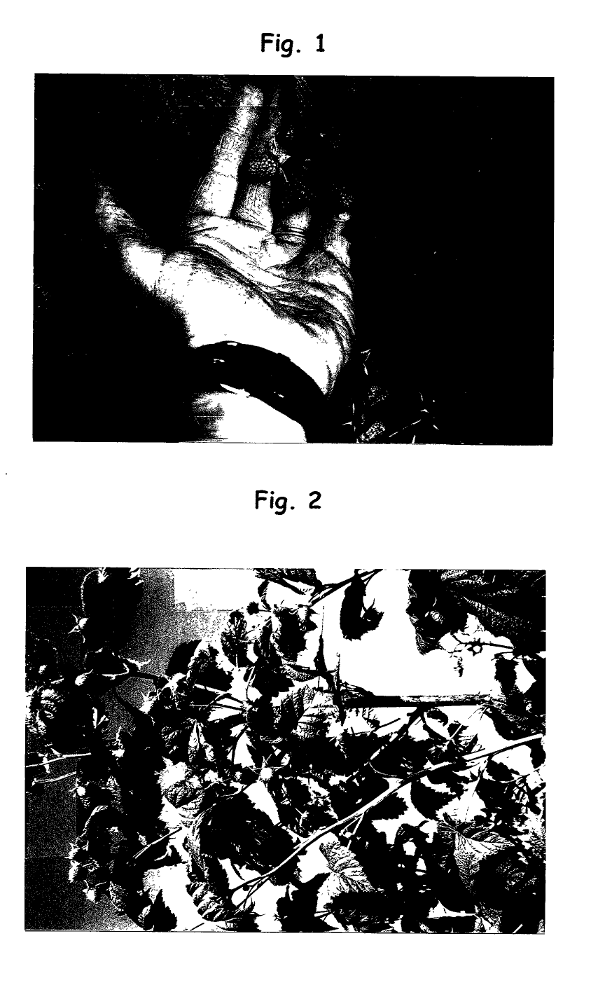

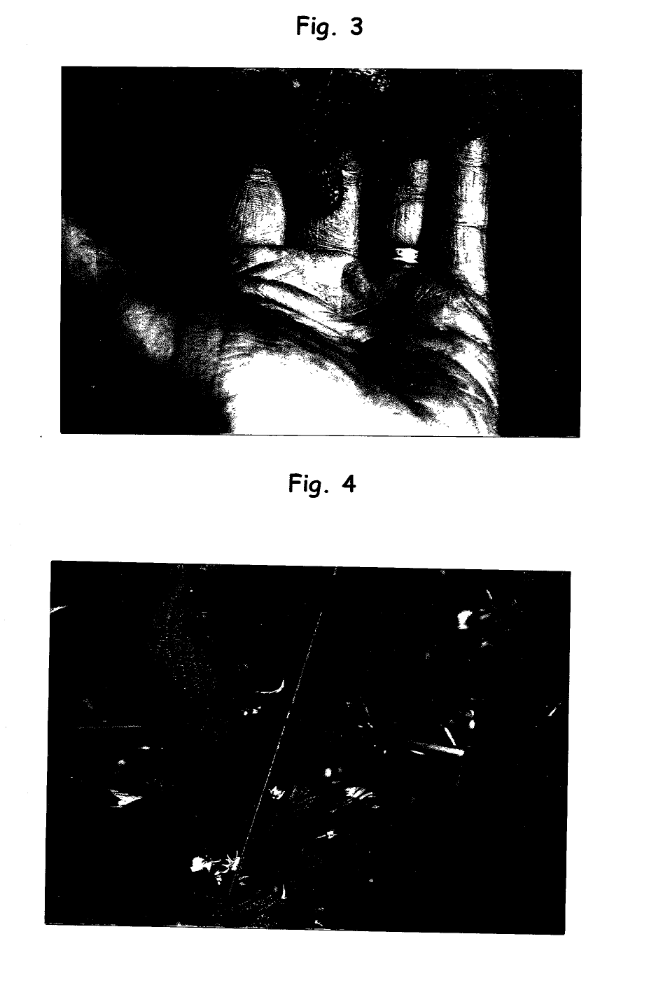

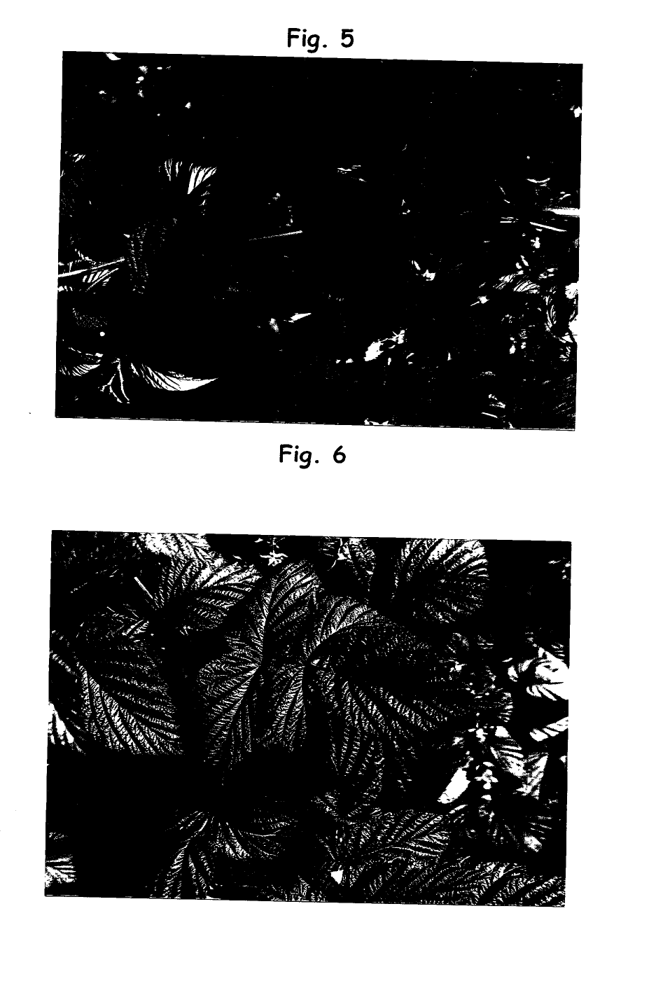

### DETAILED DESCRIPTION OF THE PREFERRED EMBODIMENT

The present invention or discovery relates to a new and distinct variety of red raspberry.

Canes: Particular features of the present invention are the young canes can grow up to 2.0 meters in height.

Fruit: The fruit color is medium red, downey in appearance and dull. The plant produces medium to large raspberries that range from 1.5-3.5 grams where the average size is about 3.0 g. The fruit length for/width ratio is substantially conical with a regular outline. Further, there a small percentage of fruit have split receptacles. The production of fruit occurs in late summer or early autumn.

Leaves and Foliage Characteristics: Autumn Byrd plants show bronzing of young leaves. Their petioles are purple at the base; leaflets are nearly sessile on the petiole (petiolule nearly absent). The spines are clear, with red pigmentation at the tip only. They are of medium density and very short. Stipules are slender, upright and short. Stems are green, with reddening near bases. The wax bloom is moderate.

Flowers: Flowers are white, self-fertile and have 5 to 6 petals per flower. Each flower produces ample pollen for good pollination. Flowers and fruit are well exposed for easy access to picking.

Disease Resistance: The present species has exhibited resistance to biotypes 1-4 of the large raspberry aphid, Agedophoten idaei. The breed is susceptible to Phytophthora root rot in glasshouse tests. It was slow to become infected with raspberry Leslie dwarf virus where it did not show distinct RBSV yellow symptoms.

While the invention is susceptible of various modifications and alternative forms, specific embodiments thereof have been shown by way of example in the drawings and will herein be described in detail. It s should be understood, however, that it is not intended to limit the invention to the particular forms disclosed, but, on the contrary, the intention is to cover all modifications, equivalents and alternatives falling within the spirit and scope of the invention as expressed in the appended claims.

I claim:

1. A new and distinct variety of red raspberry which can withstand and thrive in both Maritime Northwest and coastal California climates and which is unusually hardy and vigorous, disease resistant and a heavy producer of large crops of firm, large, sweet, juicy raspberries of deep red color and good flavor, substantially as herein shown and described and identified by characteristics described above.
